(19) United States
(12) Patent Application Publication
(10) Pub. No.: US 2010/0198324 A1
(43) Pub. Date: Aug. 5, 2010

(54) Thermo/IC cross-training suite (gel), thermo/IC cross-training suite (solid)

(76) Inventor: Leonard Charles Graaham, JR., Valley Village, CA (US)

Correspondence Address: Leonard Graham, 263 W. Olive Avenue # 308, Burbank, CA, 91502, US

(21) Appl. No.: 12/322,289

(22) Filed: Feb. 2, 2009

(51) Int. Cl. A61F 7/00; A41D 31/00

(52) U.S. Cl. 607/112; 2/243.1

(57) ABSTRACT

This system provides temporary heating and cooling of the body parts its applied to, one of the main focal points is that the design of these suites can range from physical activity to relaxing and the suites also have medical applications.

### BACKGROUND

(A) The Field of Invention

This invention relates to Sports Apparel and also has related applications in Sports Medicine in the areas of preventive and rehabilitative capabilities.

(B) Description of Related Art

In previous patent applications we have Seen areas of the body covered for rehabilitation, but not preventive, rehabilitation, and temporal climate control simultaneously in a large area, this invention will address this problem the sizes will range from small to extra large, men, women, and kids.

This invention relates to the production of patent specifications and patent applications.

### CROSS-REFERENCE TO RELATED APPLICATIONS

Not Applicable

### STATEMENT REGARDING FEDERALLY SPONSORED RESEARCH AND DEVELOPMENT

Not Applicable

### REFERENCE TO A SEQUENCE LISTING A TABLE, OR A COMPUTER PROGRAM LISTING

Not Applicable

### BACKGROUND OF THE INVENTION

In accordance with patent application background of the invention 35.USC.111 (a) there will be an Abstract Of The Disclosure behind the claims section For compliance with 37.CFR.1.72

### BRIEF SUMMARY OF THE INVENTION

The THERMO/IC CROSS-TRAINING SUITE is a sport appeal equipment, that is designed for multi use. Its primary function is temporary heating and cooling of the body or parts it is applied to, its capabilities range from physical activities such as training, or in a sporting event, when you work out or for rehabilitation purpose's and in some cases at work, or just temporary climate control.

### BRIEF DESCRIPTION OF THE SEVERAL VIEWS OF THE DRAWING

FIG. 1. Illustrates a representation of an example of the front view, inside of the suite as it appears cooled, exhibit 12 is a non-toxins propylene glycol based gel for temporary heating and cooling of the muscles, note; exhibit 12 can also be heated by a microwave oven and cooled by a freezer, exhibit 22 is a inner sleeve made of blended fabrics or nylon for holding 12 gel packs in a stationary position for temporary heating and cooling of the muscles

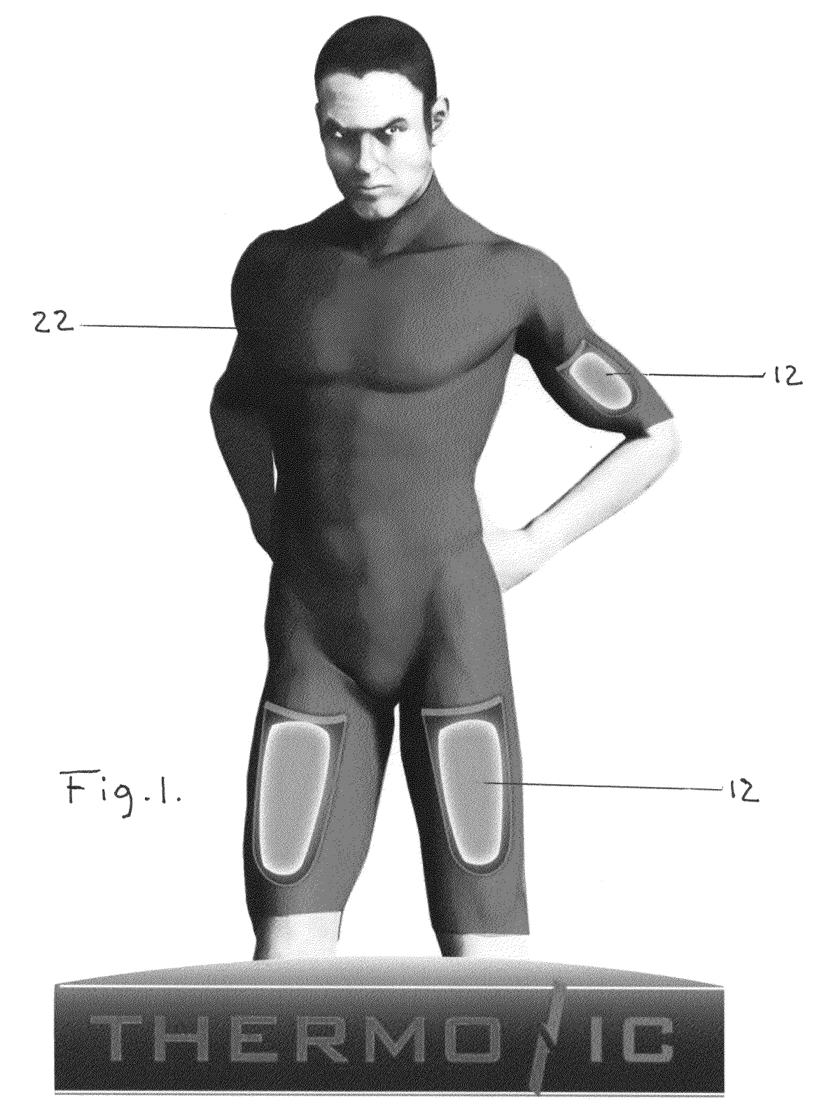

FIG. 2. Illustrates a representation of an example of the back view of the inside of the suite as it appears hot 12 B is a non toxins propylene glycol based gel for temporary heating and cooling of the muscles it is applied to, note; 12 B can be heated by a microwave oven or cooled by a freezer 22 B is a inner sleeve made of blended fabrics for holding 12 B gel packs in a stationary position for temporary heating and cooling of the muscles.

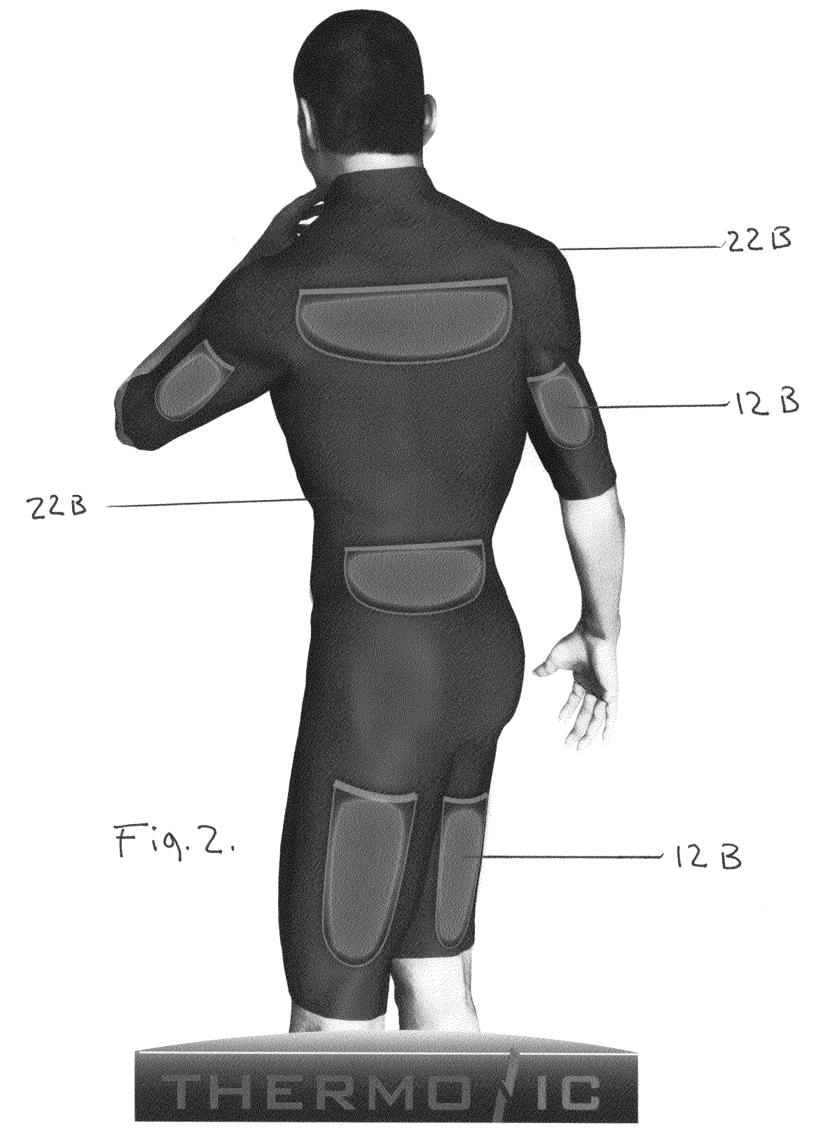

FIG. 3. Illustrates a representation of an example of the front view, outer appearance, exhibit 1 is fabrics put together to form the outward appearance example

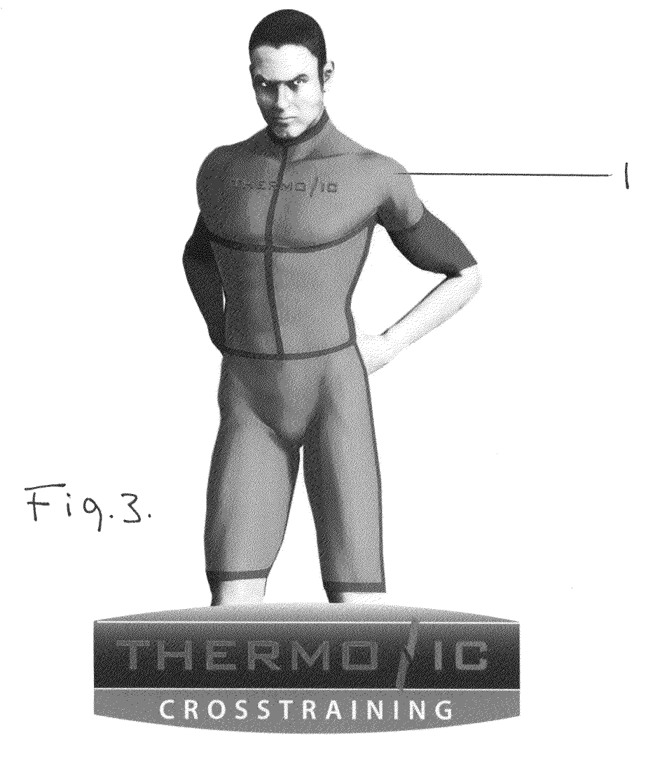

Spandex Lycra, Neoprene-etc.

FIG. 4. Illustrates a representation of the back view of the outward appearance. Of the THERMO/IC Cross-Training Suite (Gel) 1 B

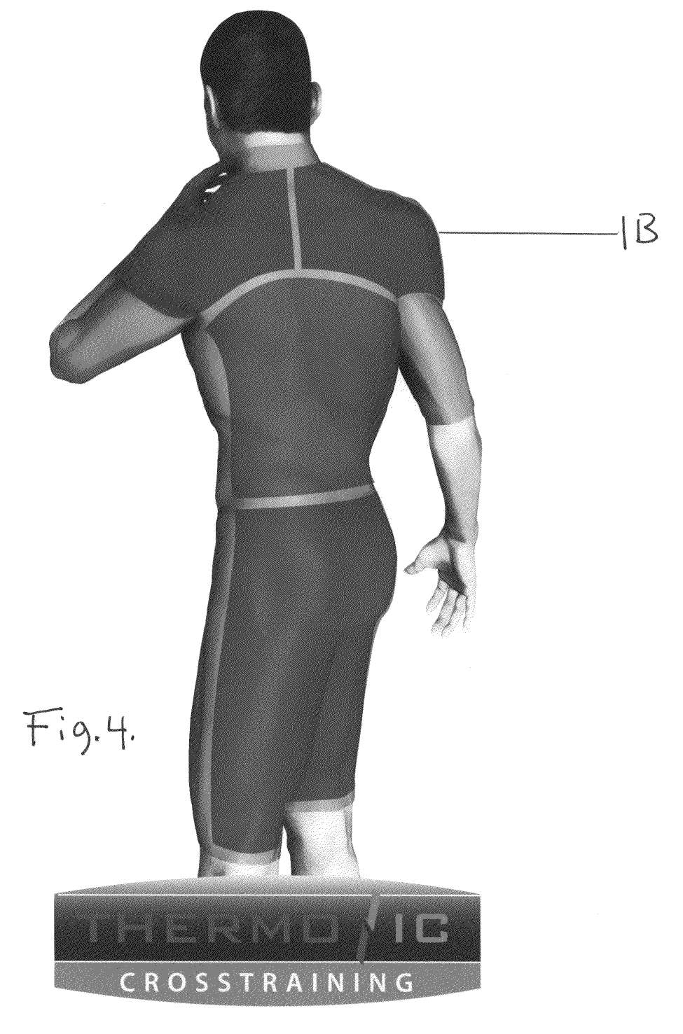

FIG. 5. Illustrates a representation of an example of exhibit 2 a solid gel pack for temporary heating and cooling of the muscles, these packs are solid and flexible and can be attached any where on the suite, on the reverse side of the pack are hooks that will allow the packs to be attached to 11 the inner sleeve that is a blended fabric of mesh for support of 2 solid gel pack, note; 2 can be heated by a micro wave oven or cooled by a freezer.

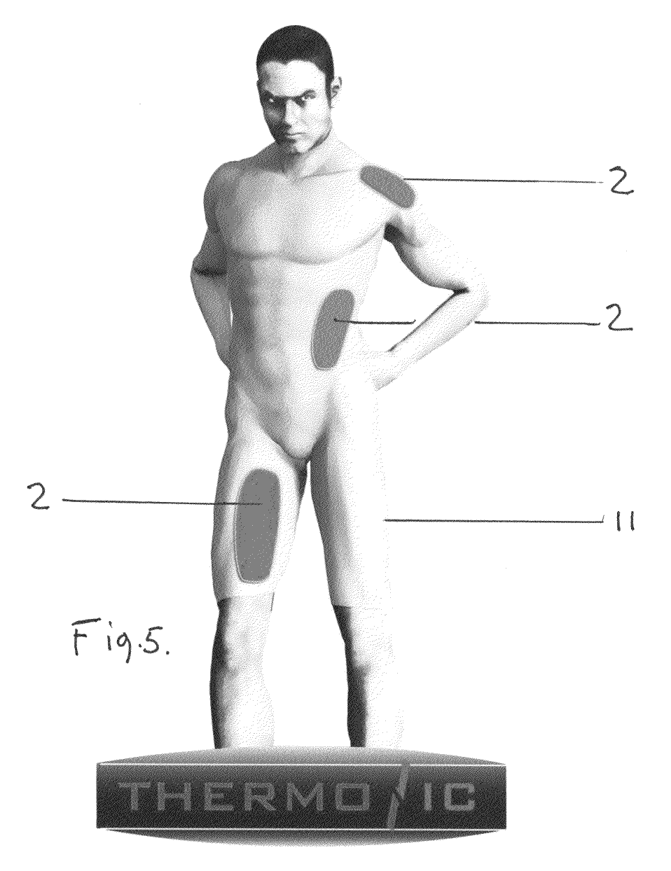

FIG. 6. Perspective view of (2) here is the back view of 2 B that shows the hooks that will attach the solid gel packs any where on (11) the inner sleeve FIG. 6. Also shows the correlation between 2 B to 11 B hooks to mesh.

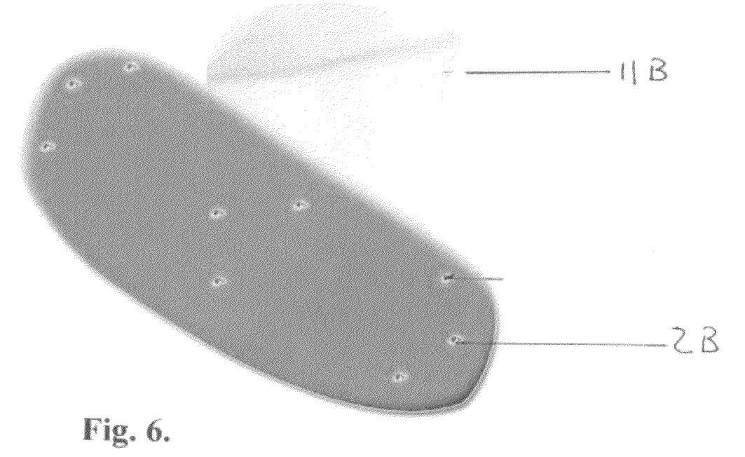

FIG. 7. shows a close up view of (2) and (11) FIG. 7. 2 C as it appears cooled is attached to the left shoulder of 11 C.

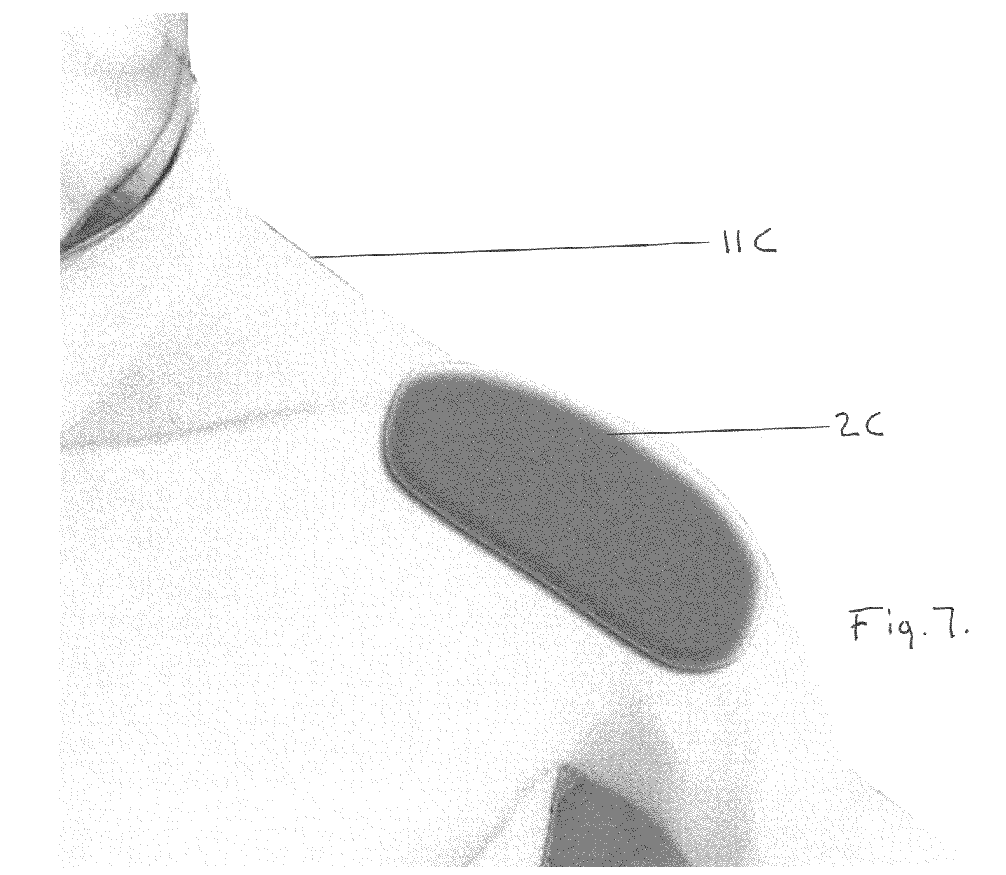

FIG. 8. Illustrates a representation of an example of the Thermo/IC Cross-Training Suite (Solid) 7 outward appearance.

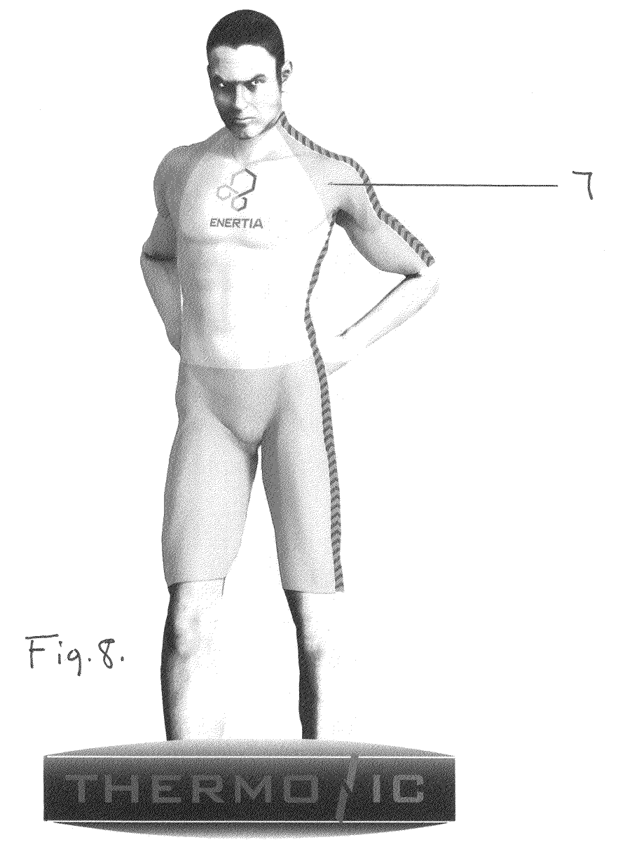

FIG. 9. Illustrates a representation of an example of the back view 7 B.

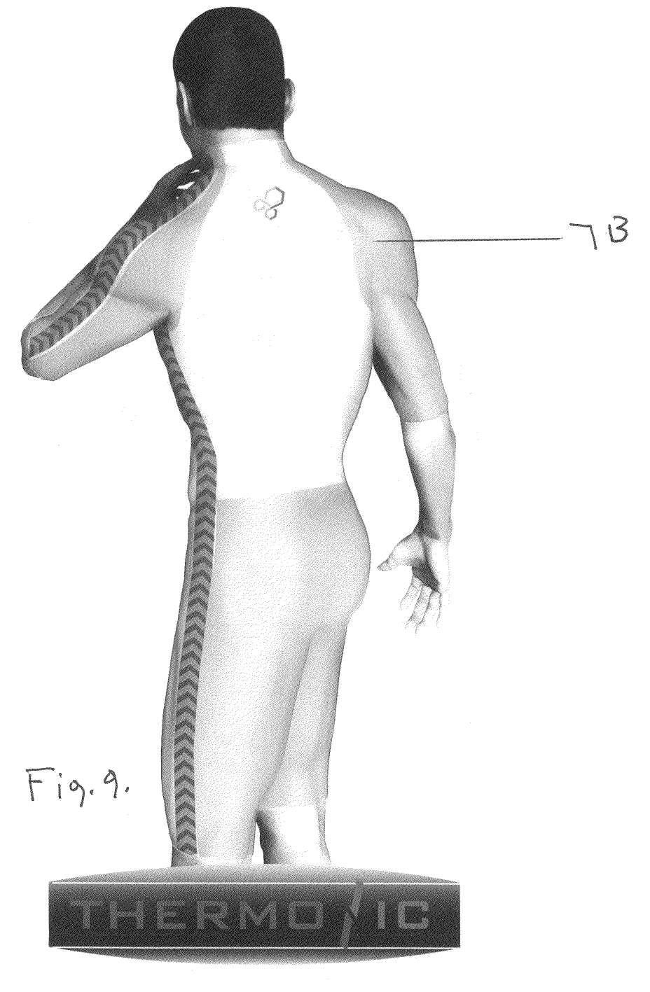

What is claimed is:

1. Two distinct suits that perform the same function, temporary heating and cooling of the body parts it is applied to, for comfort rehabilitation and preventative measures. THERMO/IC SUITE GEL (1) and THERMO/IC SUITE SOLID (7) are the products for this claim.

2. In suite (1) the therapeutic packs (12) can be heated or cooled and placed in (22) the inner sleeve in a stationary position and used for related use.

3. In suite (7) the therapeutic packs (2) a flexible solid can be heated or cooled and placed any where on (11) the inner sleeve.

4. The actual suite itself is made of different fabrics woven and or compressed together to form an viable product. Layer (A) Cotton Nylon Blend/Micromesh 0.5 mm to 3 .mm Layer (B) Neoprene 0.5 mm to 3 .mm layer (C) Spandex Lycra 0.05 mm.
